(19) United States
(12) Patent Application Publication
(10) Pub. No.: US 2008/0281170 A1
(43) Pub. Date: Nov. 13, 2008

(54) Method for Detecting Critical Trends in Multi-Parameter Patient Monitoring and Clinical Data Using Clustering

(75) Inventors: Larry J. Eshelman, Ossining, NY (US); Xinxin (Katie) Zhu, Croton-On-Hudson, NY (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 12/092,986

(22) PCT Filed: Oct. 17, 2006
(86) PCT No.: PCT/IB2006/053822

§ 371 Date: May 8, 2008

Related U.S. Application Data
(60) Provisional application No. 60/734,733, filed on Nov. 8, 2005.

(51) Int. Cl. A61B 5/00

(52) U.S. Cl. 600/301

(57) ABSTRACT

A physiological data analysis component (10) determines a condition of an individual. The physiological data analysis component (10) includes an input component (12) that receives a plurality of different physiological parameters of the individual. A classification component (20) of the physiological data analysis component (10) maps these parameters to a multi-dimensional space having a plurality of regions corresponding to two or more conditions. The classification component (20) determines the condition of the individual based on the region the physiological parameters mapped within. An output component (24) of the physiological data analysis component (10) conveys the condition of the individual to a user of the physiological data analysis component (10).

The following relates to patient monitoring and diagnosing systems. It finds particular application to analyzing multiple physiological parameters in multi-dimensional space to determine a physiological condition and/or predict a subsequent physiological condition of an individual.

Patients typically are connected to a plurality patient monitoring devices that continuously or periodically measure a variety of physiological data such as heart rate, blood pressure, blood oxygen level, core body temperature, heart electrical activity, etc. From this data as well as other data from blood analyses, bone analyses, excretion (e.g., urine, mucus, etc.) analyses, hormone analyses, etc., clinicians often determine a condition of the patient. Clinicians also use this data to predict whether the condition of the patient is remaining in or moving toward a condition (e.g., the condition is improving) or unstable condition (e.g., the condition is declining), including identifying one or more likely unstable conditions (e.g., sepsis, pancreatitis, pulmonary edema, etc.).

Conventional techniques for determining the condition of a patient include thresholding a linear combination of the physiological data. For example, a temperature may be compared to a range of “normal” temperatures, a pulse may be compared to a range of “normal” heart rates, etc. Such systems include Acute Physiology and Chronic Health Evaluation (APACHE), Simplified Acute Physiology Score (SAPS), Pediatric Risk of Mortality (PRISM), Pediatric Index of Mortality (PIM), and the like. However, physiological data usually interact in a nonlinear fashion. Systems based on linear methods fail to take into account these interactions, which are often a better indicator of the condition of the patient relative to absolute values of individual parameters or a set of parameters. In addition, these systems typically do not analyze trends in the physiological data. Systems that do analyze physiological trends commonly only analyze individual parameters. For example, electrocardiogram (ECG) monitors traditionally only analyze ECG signals over time.

With conventional techniques, nonlinear methods for analyzing multi-parameter trends over time tend to be very complex and computationally intractable.

In one embodiment, a physiological data analysis component that determines a condition of an individual is illustrated. The physiological data analysis component includes an input component that receives a plurality of different physiological parameters of the individual. The physiological data analysis component further includes a classification component that maps these parameters to a multi-dimensional space that has a plurality of regions corresponding to two or more conditions. The classification component determines the condition of the individual based on the region the physiological parameters mapped within. An output component of the physiological data analysis component conveys the condition of the individual to a user of the physiological data analysis component.

One advantage includes determining a present condition of an individual from multiple physiological parameters.

Another advantage resides in predicting a future condition of the individual from a plurality of sets of physiological parameters obtained at different time intervals.

Another advantage lies trending multiple physiological parameters over time to infer a future condition of the individual.

Still further advantages will become apparent to those of ordinary skill in the art upon reading and understanding the detailed description of the preferred embodiments.

The present technique can take form in various elements or steps and in various combinations thereof. The drawings are only exemplary of selected embodiments and are not to be taken as limiting the invention.

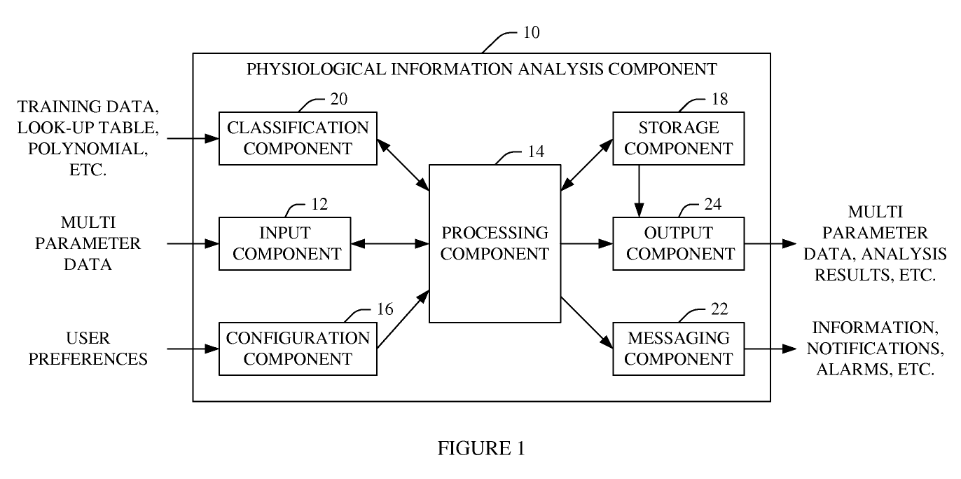

FIG. 1 illustrates a component that analyzes physiological data in multi-dimensional space to determine a present condition and/or predict a subsequent condition of an individual.

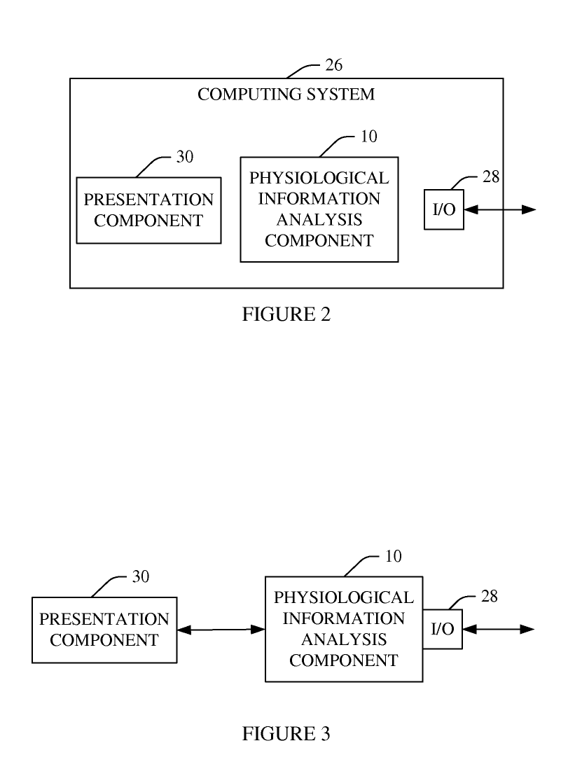

FIG. 2 illustrates a computing system in which the physiological analysis component can be employed.

FIG. 3 illustrates the physiological analysis component as an independent device.

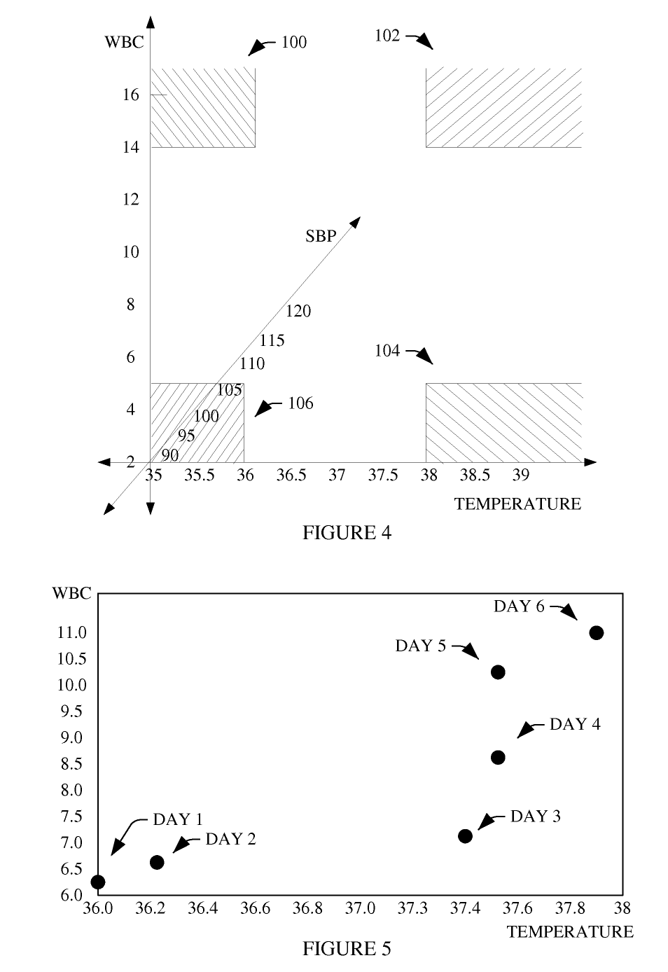

FIG. 4 illustrates an exemplary mapping of regions indicative of sepsis within multi-dimensional space used to determine a present condition of an individual.

FIG. 5 illustrates an exemplary trend of physiological parameters in multi-dimensional space used to predict a future condition of an individual.

FIG. 1 illustrates a physiological data analysis component 10 that analyzes physiological data in multi-dimensional space to determine a present condition of an individual and/or predict a subsequent condition of the individual. Examples of suitable physiological data include, but are not limited to, heart rate, blood pressure, blood oxygen level, core body temperature, heart electrical activity, white blood count, hormone level, etc. For determining and predicting the condition of the individual, stable conditions and unstable conditions, such as sepsis, are modelled within multi-dimensional space. In a preferred embodiment, this is achieved by mapping physiological parameters indicative of particular conditions (stable and unstable) to the multi-dimensional space and correspondingly labelling those regions within the multi-dimensional space (or assigning a degree of severity—i.e., a severity metric). To determine the present condition of the individual, physiological parameters from the individual are mapped to the multi-dimensional space. The condition of the individual is determined based at least in part on the region in which the physiological parameters are mapped. To predict a future condition, a plurality of sets of physiological parameters of the individual obtained over time are mapped to the multi-dimensional space. A trend based on two or more of the mappings is used to infer the future condition of the individual.

The analysis component 10 includes an input component 12 that receives the physiological data such as parameters representative of heart rate, blood pressure, blood oxygen level, core body temperature, heart electrical activity, white blood count, hormone level, etc. In one instance, the input component 12 is coupled (e.g., via a data port) to one or more physiological monitoring devices (e.g., ECG monitor, blood pressure monitor, thermometer, etc.) that sense physiological data and convey the sensed physiological data to the analysis component 10 through the input component 12. It is to be appreciated that such physiological data can be raw or processed data. Additionally or alternatively, the input component 12 includes wired and/or wireless network componentry (not shown) for receiving physiological data over a network, including the Internet. For example, the input component 12 can receive physiological data from sensors residing in a body area network (BAN), a database, a server, a physiological data monitor, a computer, another physiological data analysis component, a cell phone, a personal data assistant (PDA), email, a message store, etc. Additionally or alternatively, the input component 12 includes a port for receiving portable storage (e.g., various types of flash memory, CD, DVD, optical disk, cassette tape, etc.), which can be used to transfer physiological data to the analysis component 10. Additionally or alternatively, the input component 12 can be attached to a keyboard, a keypad, a touch screen, a microphone, or other input device and receive physiological data through such devices, for example, from a user.

A processing component 14 controls the input component 12. The processing component 14 can access a configuration from a configuration component 16 to determine a frequency in which the input component 12 accepts physiological data. It is to be appreciated that the frequency can be defined by a user and/or automatically determined based on historical activity, probabilities, inferences, user identification, etc. In one instance, the configuration defines a polling frequency, wherein the input component 12 polls other devices (e.g., monitoring devices, computers, databases, etc.) to determine whether physiological data is available. Such polling can be through a uni-cast to a particular device, a multi-cast to a group of devices, and/or a broadcast to any device with componentry and permission to communicate with the analysis component 10. In another instance, the configuration may determine that the analysis component 10 should enter an idle or sleep state when physiological data is not available and a wake state when physiological data becomes available. The device delivering the physiological data can send a notification and wait for the analysis component 10 to wake up and respond (e.g., go ahead and send the data, do not send any data, etc.) or it can simply emit the physiological data.

The processing component 14 stores received physiological data in the storage component 18. The stored data can include raw and/or processed data and can be associated with information such as an identity of the individual, a time stamp, a medical history of the individual, a type of data (e.g., temperature, blood pressure, etc.), an identity of the source of the data, etc. Additionally or alternatively, external storage (not shown) is used. For example, external storage can be used to provide a greater volume of storage. In another example, external storage can be used to reduce storage requirements and/or the footprint of the analysis component 10. In yet another example, external storage is used as a redundant back-up system.

The configuration component 16 also includes instructions on how the processing component 14 should process the data. For instance, the instructions can indicate which types (e.g., ECG, temperature, blood analysis, etc.) of data to use in a particular analysis. For example, the user may decide to limit the types of data and/or number of types analyzed in order to reduce processing time. In another example, the user may desire to mitigate using particular types of data deemed to provide little or no value in determining the condition of the individual. The instructions may also indicate a number of data points to use in a particular analysis. For example, the instructions may indicate that a week's worth of data should be captured prior to using the data to determine a present or future condition. Once this amount of data is acquired, the processing component 14 retrieves and analyzes the data.

A classification component 20 determines the present and/or anticipated future condition of the individual based on the received physiological information. As described above, this can be achieved by mapping physiological parameters indicative of particular conditions to multi-dimensional space from many individuals and labelling those regions. Physiological parameters from the current individual are mapped into the labelled multi-dimensional space. For instance, physiological data representative of a “normal,” or stable state can be used to define regions within the multi-dimensional space, wherein an individual is deemed “normal” if his/her physiological data falls within any of these regions. Physiological data representative of “abnormal,” or unstable states can be used to define regions of instability (e.g., sepsis) within the multi-dimensional space. An individual is deemed as having the condition associated with the region in which his/her physiological data falls within. By way of example, physiological parameters indicative of sepsis can be mapped to one or more regions within the multi-dimensional space, which regions are labelled as sepsis. If the physiological data of the individual is mapped to any of these regions, the individual is deemed likely to have sepsis. It is to be appreciated that regions for different conditions may overlap. In such situations, the individual can be deemed as likely to be associated with one or more of the conditions. Further analysis can be performed to reduce the number of potential conditions, if possible.

Subsequent measurements of physiological parameters are preferably mapped to facilitate predicting the future condition of the individual. For instance, a trend based on two or more of the mappings obtained at different time intervals is used to infer the future condition of the individual. For instance, the trend is used to determine whether the individual is likely to remain in a “stable” region; move from a “stable” region to an “unstable” region (e.g., representing a decline in health); remain within an “unstable” region; move from one “unstable” region to another “unstable” region; and move from an “unstable” region to a “stable” region (e.g., representing an improvement in health). By way of example, if a trend of the individual's physiological data shows a progression toward a sepsis region, it can be inferred that the individual may have or may be about to develop sepsis.

The data points used for trending are determined by the configuration component 14. For example, if physiological data is received and stored daily, the configuration component 14 may deem each day a data point. Of course, other time increments are also contemplated, e.g. hourly. A vector is generated between each data point (or data from each day), and a resultant vector over a number of days, or data points, projects the future condition of the individual. Additionally or alternatively, each individual vector is analyzed to determine the future condition of the patient. Furthermore, the data points are used to predict the future condition through extrapolation, which extrapolation is used to predict a mapping of subsequently measured physiological parameters.

Depending on the type and source of data, the data acquired within each time interval may be different. For instance, temperature may be continuously measured through a rectal probe, blood pressure may be measured hourly through a non-invasive technique, white blood cell count may be determined daily, etc. Such data can be variously rolled up. For example, the temperature can be average over the day or some subset of time, including multiple averages throughout a single day. For instance, temperature may be averaged hourly and used along with the hourly blood pressure measurements during analysis. In another example, the temperature and the blood pressure is averaged over the day and the average is used along with the daily white blood cell count during analysis.

The classification component 20 preferably executes one or more classification or regression algorithms on combinations of data reflective of known conditions in order to label regions within the multi-dimensional space and/or on physiological data in order to map measured physiological parameters to the multi-dimensional space and to label the patients condition or assign a severity metric. Suitable techniques, algorithms, approaches, schemes, etc. include using one or more of the following: neural networks (e.g., multi-layered perceptrons, radial basis functions), expert systems, fuzzy logic, support vector machines, Bayesian belief networks, etc. Furthermore, the mapping can be done through one or more look-up tables and/or expansion of a polynomial representative the multi-dimensional space. Moreover, the classification component 20 can be developed or trained using various methods, including a priori knowledge, various clustering techniques (e.g., k-means, k-medoids, hierarchical methods, Expectation Maximization (EM)), probabilistic and/or statistic-based analysis and pattern recognition techniques, or techniques associated with the specific classifier used (e.g., backpropagation for a multi-layered perceptron). The training algorithm would use known unstable conditions and associated parameters, known stable conditions and associated parameters, ranges of parameters typically associated with stable conditions, results from analysis, etc.

A messaging component 22 provides a mechanism in which the analysis component 10 notifies clinicians, applications, devices, bed side monitors, etc. For instance, the configuration component 16 may indicate that the analysis component 10 should only transmit a notification when an individual is moving from a stable (e.g., normal, known condition, etc.) state toward an unstable (e.g., life threatening, abnormal, etc.) state. As such, the analysis component 10 can execute in connection with monitoring devices and/or subsequently process physiological data and inform one or more clinicians when the individual is becoming unstable. In another instance, the configuration component 16 indicates that the analysis component 10 should only transmit a notification when an individual is moving from an unstable state to a stable state. In yet another instance, the configuration component 16 indicates that the analysis component 10 should only transmit a notification upon any change in state, including moving from one unstable state to another unstable state. The messaging component 22 can use various communication schemes to provide such notices. For instance, the messaging component 22 triggers an audible and/or a visual alarm at a bed side or central monitoring station. In another instance, the messaging component 22 notifies a clinician through one or more of a conventional telephone, a cell phone, a pager, email, a PDA, etc. An output component 22 enables the analysis component 10 to convey collected and/or processed data and/or results to clinicians, applications, devices, etc.

FIG. 2 illustrates a computing system 26 in which the physiological analysis component 10 can be employed. The computing system 26 can be essentially any machine with a processor. For instance, the computing system 26 can be a bed side monitor, a desktop computer, a laptop, a personal data assistant (PDA), a cell phone, a workstation, a main frame computer, a hand held computer, a device for measuring one or more physiological states of an individual, etc. The analysis component 10 can be implemented in hardware (e.g., a daughter or expansion board) and/or software (e.g., one or more executing application) in connection with the computing system 26.

The computing system 26 includes various input/output (I/O) component 28. For instance, the computing system 26 includes interfaces for receiving information from one or more of the following: a keyboard, a keypad, a mouse, a digital pen, a touch screen, a microphone, radio frequency signals, infrared signals, portable storage, etc. The computing system 26 also includes interfaces for presenting. For instance, the computing system 26 includes interfaces to various printing, plotting, scanning, etc. devices. The computing system 26 further includes interfaces for conveying information. For example, the computing system 26 includes wired and/or wireless network interfaces (e.g., Ethernet, etc.), communication ports (e.g., parallel and serial), portable storage, etc. A presentation component 30 is used for displaying data, prompting a user for input, interacting with a user, etc. Suitable displays include liquid crystal, flat panel, CRT, touch screen, plasma, etc. Also, a danger light or audio alarm can be sounded.

By way of example, the I/O component 28 receives the physiological data used to generate the model and map physiological parameters of an individual to the model. This data is conveyed to the analysis component 10 and mapped to a multi-dimensional model as described above. The model defines regions which are associated with particular conditions based on physiological parameters. The regions are accordingly labelled as stable or instable, including the particular condition (e.g., sepsis), or assigned a value on severity metric. Alternatively, once a suitable map is determined, the map is directly loaded into analysis devices. An individual's present condition is determined by mapping physiological parameters of the individual to one or more regions defined within the multi-dimensional space and obtaining the corresponding condition labels. A future condition is predicted by trending physiological parameters of the individual over time and inferring the future condition from the trend. The model, individual points, and/or results can be presented via the presentation component 30 and/or conveyed to a clinician, an application, a device, etc. through the I/O component 28.

FIG. 3 provides an example in which the physiological analysis component 10 is an independent device. In this example, the analysis component 10 includes the input/output (I/O) component 28, which is used for receiving and/or conveying information from and/or to other components, and is connected to the presentation component 30. Similar to the above, the I/O component 28 receives the physiological data used to generate the model and map physiological parameters of an individual to the model and conveys results and/or data, and the presentation component 30 presents the results and/or data. The analysis component 10 defines regions of stability and instability within multi-dimensional space and maps one of more sets of physiological parameters to determine the condition and/or future condition of the individual as described in detail above.

FIGS. 4 and 5 illustrate non-limiting examples for determining a present and/or future condition of an individual. In these examples, the condition is sepsis. However, it is to be understood that essentially any condition, stable or unstable, can be mapped to the N dimensional space. Suitable parameters for detecting the onset of sepsis include, but are not limited to, body temperature, heart rate, respiration rate, systolic blood pressure, and white blood cell count. Exemplary parameter values that are indicative of sepsis include the following:
Body Temperature (T): >38° C. or <36° C.;
Heart Rate (HR): >90 beats/min;
Respiration Rate (RR): >20 breaths/min, or PaCO 2<32 mmHg;
Systolic Blood Pressure (SBP): <90 mmHg, or Mean Arterial Pressure <65 mmHg; and
White Blood Cell count (WBC): >12,000 or <4000 cells/microliter.

Parameters like WBC can be further delineated into various constituent components, which may be associated with the following “normal” ranges:

Neutrophils: 50-70%, or 7.4-10.4 thousand/cu.mm;

Lymphocytes: 20-30%;

Monocytes: 1.7-9%;

Eosinophils: 0-7%; and

Basophils: <1%.

FIG. 4 illustrates portions of regions within N dimensional space, wherein N is an integer equal to or greater then one, that are indicative of sepsis based on a subset of the above criteria. Only three (WBC, T, and SBP) of the above criteria are illustrated for purposes of clarity. However, it is to be appreciated that other combinations with more, the same, or less criteria, including different criteria, are contemplated. As depicted in FIG. 4, white blood cell count represents one dimension, temperature represents another dimension, and systolic blood pressure represents yet another dimension. The particular axis for any parameter may be arbitrary or not.

Using the ranges illustrated above, a plurality of regions 100, 102, 104 and 106 indicative of sepsis are defined within the N dimensional space, where N=3 in this example. For explanatory purposes, the regions 100-106 are illustrated as rectangular volumes. However, it is to be appreciated the regions 100-106 can be variously shaped. For example, suitable shapes include spheres, elliptical volumes, irregular volumes, etc. In addition, multiple conditions (stable and other unstable) can be defined within one or more regions in the N dimensional space, and such regions may or may not overlap. Thus, a particular region within the N dimensional space may be indicative of sepsis, sepsis and one or more other unstable conditions, at least one other unstable condition, or a stable condition.

A present condition of an individual is determined by analyzing similar parameters associated with the individual and mapping the set of parameters in the N dimensional space. If the parameters map to a region labeled as sepsis, the individual is deemed likely to have sepsis. If the parameters map to a region labeled as stable (not shown), the individual is deemed likely to be stable. If the parameters map to a region with more than one label (e.g., an overlapping region), the individual is deemed likely to be associated with one or more conditions (not shown). For any point in the N dimensional space, a metric can be assigned in order to represent a severity or likelihood of a condition.

FIG. 5 illustrates a non-limiting example for predicting a future condition of the individual by tracking one or more of the N physiological parameters and determining which regions within the N dimensional space the parameters are moving towards. In this example, only two (WBC and temperature) of the above parameters with respect to time are illustrated for sake of clarity. However, it is to be appreciated that other combinations with more, the same, or less criteria, including different criteria, are contemplated.

In a preferred embodiment, a time-series analysis is used to determine the likelihood that at a next increment of time the individual will be associated with one or more particular conditions based on one or more movements within the N dimensional space. In this example, the condition of the individual is depicted over six days as follows: on a first day (“DAY 1”), the N parameters of the individual map to a point at 112 in the N dimensional space; on a second day (“DAY 2”), the N parameters of the individual map to a point at 114 in the N dimensional space; on a third day (“DAY 3”), the N parameters of the individual map to a point at 116 in the N dimensional space; on a fourth day (“DAY 4”), the N parameters of the individual map to a point at 118 in the N dimensional space; on a fifth day (“DAY 5”), the N parameters of the individual map to a point at 120 in the N dimensional space; and on a sixth day (“DAY 6”), the N parameters of the individual map to a point at 122 in the N dimensional space.

An expected severity of a condition of the individual at a next increment of time, a day in this example, can be determined by taking the product of a metric of severity of any point in the N dimensional space and a likelihood or confidence that the individual will be in that region of the space at the next time increment. This is preferably achieved through a time series analysis. The particular time series algorithm used may be based on the nature of the problem or otherwise. In one instance, a traditional linear model, such as an Autoregressive Moving Average model (ARMA), is used. In other instances, a nonlinear model (e.g., a neural network using a window in time, a recurrent neural net with feedback, etc.) is used.

A number of points used for predicting a next point in time can be selected by the user. Each time step is preferably analyzed as a vector in which a set of recent time-step vectors is used to predict the next vector (e.g., a direction of the next step) or determine a likelihood or confidence that the individual will be in some neighboring region of the N dimensional indicator space. The step size and/or step weighting can vary depending upon the application of otherwise. For instance, for sepsis a window of several days might be appropriate.

Various techniques can be used when employing parameters sampled at different rates (e.g., temperature may be sampled every hour whereas WBC may be measured every 8 hours). For example, for the parameter with a relatively greater sampling rate, the samples closer in time to the less sampled parameters can be used. In another example, a period in which there is at least one sample for each parameter (e.g., a day) can be selected. For the parameters associated with multiple samples, a mean or median value can be used.

Table 1 illustrates exemplary data for an individual progressing toward sepsis. The time step is in days over a six day period. The data for each day includes a representative (e.g., mean, median, absolute, etc.) value for each parameter. Using a time-series analysis, the data from all six days or a subset thereof is used to determine a likelihood that the individual on a subsequent day will be in various neighboring states in the N dimensional space. An assessment of an expected severity determines whether to invoke a pro-active intervention.

TABLE 1
Exemplary data for an individual progressing towards sepsis.

| Signs and | | | | | | |
|---|---|---|---|---|---|---|
| Symptons | Day 1 | Day 2 | Day 3 | Day 4 | Day 5 | Day 6 |
| Temperature | 36 | 36.2 | 37.4 | 37.5 | 37.5 | 37.9 |
| SBP | 125 | 120 | 120 | 105 | 103 | 100 |
| MAP | 90 | 92 | 89 | 76 | 72 | 70 |
| HR | 66 | 68 | 80 | 77 | 89 | 88 |
| RR | 14 | 14 | 15 | 16 | 17 | 20 |
| WBC | 6.05 | 6.5 | 6.95 | 8.79 | 9.8 | 10.92 |
| Neutrophils | 5 | 5.2 | 5.5 | 6.9 | 7.5 | 8.4 |
| Lymphocytes | .8 | .9 | .92 | .95 | 1 | 1.1 |
| Monocytes | .2 | .27 | .33 | .56 | .78 | .8 |
| Eosinophils | .04 | .09 | .13 | .29 | .41 | .5 |
| Basophils | .01 | .04 | .07 | .09 | .11 | .12 |

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A physiological data analysis component that determines a condition of an individual, comprising:
an input component that receives a plurality of different physiological parameters of the individual;
a classification component that maps the plurality of physiological parameters to a multi-dimensional space having a plurality of regions corresponding to two or more conditions and determines the condition of the individual based on the region the physiological parameters mapped within; and
an output component that conveys the condition to a user of the component.

2. The physiological data analysis component as set forth in claim 1, wherein the classification component maps two or more sets of physiological parameters obtained at different time intervals and predicts a future condition of the individual based on a trend derived from the mappings.

3. The physiological data analysis component as set forth in claim 2, wherein the classification component performs a time-series analysis to determine the trend.

4. The physiological data analysis component as set forth in claim 2, wherein the classification component generates the trend by connecting two or more mappings through a vector and extrapolating subsequent mapping.

5. The physiological data analysis component as set forth in claim 2, wherein the physiological parameters mapped to the multi-dimensional space include one or more of the following:
temperature;
heart rate;
respiration rate;
systolic blood pressure; and
white blood cell count.

6. The physiological data analysis component as set forth in claim 1, wherein the classification component maps the physiological parameters to the multi-dimensional space through one or more of the following techniques: clustering, k-means, k-medoids, Expectation Maximization (EM), neural networks, hierarchical methods, probabilistic analysis, statistic analysis, a priori knowledge, classifiers, support vector machines, distance measures, expert systems, Bayesian belief networks, fuzzy logic, pattern recognition, interpolation, extrapolation, data fusion engines, look-up tables and polynomial expansion.

7. The physiological data analysis component as set forth in claim 1, wherein the physiological data includes two or more of heart rate, blood pressure, blood oxygen level, core body temperature, heart electrical activity, white blood count, and hormone level.

8. The physiological data analysis component as set forth in claim 1, wherein the classification component defines one or more regions of stability within the multi-dimensional space by mapping physiological parameters indicative of a stable condition to the multi-dimensional space and labelling these regions as stable.

9. The physiological data analysis component as set forth in claim 1, wherein the classification component defines one or more regions of instability within the multi-dimensional space by mapping physiological parameters indicative of an unstable condition to the multi-dimensional space and labelling these regions based on the unstable condition.

10. The physiological data analysis component as set forth in claim 1 wherein the unstable condition regions are predetermined for patients previously diagnosed with each unstable condition.

11. The physiological data analysis component as set forth in claim 1, further including a messaging component that transmits a notification when the condition of the individual is predicted to change.

12. The physiological data analysis component as set forth in claim 1, further including an output component for conveying at least one of collected data, processed data, and results.

13. A method for determining a condition of an individual, comprising:
receiving a plurality of physiological parameters of the individual; and
determining the condition of the individual by mapping the plurality of physiological parameters to a region in multi-dimensional space that correlates to a particular condition.

14. The method as set forth in claim 13, further comprising:
mapping at least one other set of physiological parameters obtained at a different time interval; and
predicting a future condition of the individual based on a change between the mappings.

15. The method as set forth in claim 14, wherein the change is represented as a vector progressing towards the future condition.

16. The method as set forth in claim 13, further including:
using a multi-dimensional clustering analysis to generate a vector based on the plurality of received physiological parameters.

17. The method as set forth in claim 13, further including:
defining one or more regions within the multi-dimensional space by mapping physiological parameters indicative of one or more conditions to the multi-dimensional space and labelling these regions.

18. The method as set forth in claim 13, further including:
conveying at least one of a message indicative of the condition of the individual, a message indicative of a future condition of the individual, and the physiological parameters.

19. A computer programmed to perform the method of claim 13.

20. A method for determining a present and a future condition of an individual, comprising:
identifying regions of stability and instability within multi-dimensional space;
receiving a set of physiological parameters of the individual;
determining the present condition of the individual by mapping the set of physiological parameters to the multi-dimensional space in which the condition of the individual is based on the region the physiological parameters mapped within;
receiving one or more additional sets of physiological parameters of the individual, each set obtained at a different time;
mapping the one or more additional sets of physiological parameters within the multi-dimensional space;
generating a trend based on the mapped sets of physiological parameters; and
projecting a future condition of the individual based on the trend.
